(19) United States
(12) Patent Application Publication
(10) Pub. No.: US 2021/0085739 A1
(43) Pub. Date: Mar. 25, 2021

(54) ANTIPLATELET ACTIVITY OF THE ACANTHUS MOLLIS SEEDS' TOTAL EXTRACT AND ITS CONSTITUENTS

(71) Applicants: Alexandros TSELEPIS, Ioannina (GR); Spyridon ANAGNOSTOPOULOS, Xanthi (GR); Dimitrios GEORGITSOPOULOS, Palaio Faliro Attikis (GR)

(72) Inventors: Alexandros TSELEPIS, Ioannina (GR); Spyridon ANAGNOSTOPOULOS, Xanthi (GR); Dimitrios GEORGITSOPOULOS, Palaio Faliro Attikis (GR)

(21) Appl. No.: 16/603,655

(22) PCT Filed: Apr. 10, 2017
(86) PCT No.: PCT/GR2017/000019

§ 371 Date: Oct. 8, 2019

(51) Int. Cl. A61K 36/19; A61K 31/706; A61K 31/7034; A61P 7/02

(52) U.S. Cl. CPC A61K 36/19; A61K 31/706; A61K 2236/333; A61P 7/02; A61K 31/7034

(57) ABSTRACT

Acanthus mollis seeds' methanol extract exhibits an advantageous antiplatelet activity towards AA- and TRAP-6-induced platelet aggregation, i.e., inhibits two important pathways leading to platelet aggregation (pathway mediated by TxA₂ formed from AA by the action of COX-1 as well as thrombin receptor PAR-1-mediated platelet aggregation, respectively). The antiplatelet activity towards AA is due to the DIBOA-Glc content. The DIBOA-Glc activity is enhanced in the presence of Verbascoside and/or Isoverbascoside. The antiplatelet activity towards TRAP-6 is exclusively attributed to Isoverbascoside. The constituents of Acanthus mollis seeds is a new therapeutic agent for CVD patients which can replace aspirin in aspirin resistant patients (DIBOA-Glc alone or in combination with Verbascoside or/and Isoverbascoside) and also may be used as a specific antagonist of the platelet thrombin receptor PAR-1 (Isoverbascoside). Moreover, the total extract may be used as a supplement with aspirin and a P2Y12 antagonist in some patients.

### FIELD OF THE INVENTION

The present invention relates to the antiplatelet activity expressed by the total extract of Acanthus mollis seeds as well by each one of its constituents DIBOA-Glc, Verbascoside and Isoverbascoside as well as their combination.

### BACKGROUND OF THE INVENTION

Nowadays atherothrombosis is the leading cause of death worldwide. Platelet aggregation plays an important role in the pathophysiology of artery and venous thrombosis, while platelet activation also significantly contributes to atherogenesis and other pathophysiological conditions such as inflammation and carcinogenesis. Therefore, antiplatelet therapy represents the cornerstone for the treatment of patients with cardiovascular disease (CVD) and particularly patients presenting an acute coronary syndrome (ACS). Furthermore, long-term treatment of patients with antiplatelet drugs may significantly contribute to the prevention of other disorders such as cancer. Currently available antiplatelet drugs target key points of platelet activation such as the synthesis of thromboxane A₂ (TxA₂) via cyclooxygenase-1 (COX-1), the P2Y₁₂ receptor of Adenosine Diphosphate (ADP) and the integrin-receptor α_{IIb}/β₃ (GPIIb/IIIa). Current research is directed towards the development of antagonists targeting other platelet receptors such as the thrombin receptor, named as Protease Activated Receptor-1 (PAR-1) (Michelson A D, et al. Nat Rev Drug Discov. 2010; 9:154-69; Tsoumani M E, Tselepis A D, Curr Pharm Des. 2017; Epub ahead of print)

The last years, particular interest in current research have natural products (olive oil, mushrooms, wine, etc.) and their constituents which exhibit several biological activities such as anti-inflammatory, antioxidant and antiplatelet (Kontogianni V G, et al. J. Agric. Food Chem. 2016; 22:4511-4521; Tzakos A G, et al. J Agric Food Chem. 2012; 60:6977-83; Jose N, et al. Phytother. Res. 2004; 1:43-6). Therefore, the aim of the present research was to study the possible antiplatelet effect of Acanthus mollis seeds' total extract and to identify the main components responsible for the expression of this effect.

Acanthaceae is a large family of plants comprising 4.300 species categorized to 346 genera (Rezanka T, et al. Phytochem. 2009; 70:1049-1054). The members of this family are found in tropical and temperate regions, mainly in the Mediterranean region. Acanthus is a genus of wild flowers which grows in meadows, forests and rocky and bushy hills. The most important representative of this genus is Acanthus mollis, an herbaceous, perennial plant that is found in various Mediterranean regions, from Portugal and North-West Africa to the Balkan countries. The plant germinates only during the spring and early summer. However, it is described as multiannual because the rhizome remains alive throughout the year. The main foliage is formed by lobed, slightly thorny, glossy, green leaves. A special feature of the leaves is their soft texture as they do not have thorns or fluff. The flowers are white with purple color. The fruit of the plant has a smooth surface, its size resembles that of the lemon and its color is green. As it matures, it hardens breaks and eliminates the seeds that it contains. The seeds are also hard, have brown color, a slightly crumpled coat and their shape is oval and flat. Acanthus mollis is used against psoriasis and other skin diseases that occur with an imbalanced production of eicosanoids. It was proved that the methanol leaves' extract inhibits the 5-lipoxygenase (5-LOX) and cyclooxygenase-1 (COX-1) activities at a concentration of 200 mg/mL and increases the biosynthesis of 15 (S)-HETE, an anti-inflammatory eicosanoid (Bader A, et al. Phytother. Res. 2015; 29:108-113). However, to the best of our knowledge, the effect of Acanthus mollis seeds methanol extract on human platelet activation has not been investigated so far.

### DESCRIPTION OF THE INVENTION

In the first step of the present research protocol we aimed to identify the best method of extraction of the Acanthus mollis seeds in order to obtain the extract exhibiting the best biological activity towards platelet aggregation. Therefore, 13 g of dry seeds (stored at −20° C., two days after their harvesting), were grinded-pulverized with a brass mortar into a powder. The powder was then extracted sequentially with 200 mL of two solvents of gradually increasing polarity first hexane and then methanol in a Soxhlet apparatus for 6 h with each solvent. We ended up in this extraction method after having found in preliminary experiments that among all extracts prepared (hexane extract, ethyl acetate extract, dichloromethane extract and methanol extract) obtained by extraction in a Soxhlet apparatus only the methanol extract exhibited biological activity towards platelet aggregation, whereas all other extracts had little or no antiplatelet effect.

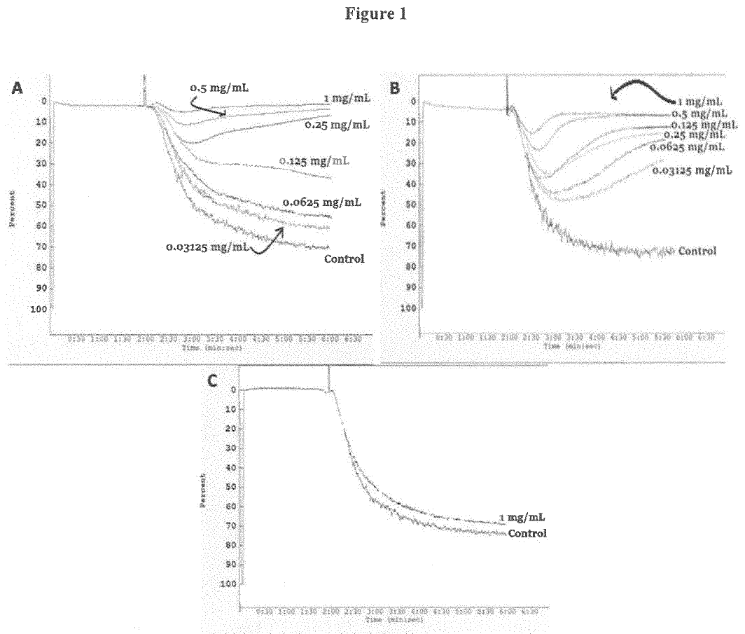

The effect of methanol extract on platelet aggregation in vitro was extensively studied in human Platelet Rich Plasma (PRP) from healthy volunteers, using Light Transmittance Aggregometry (LTA), as we previously described (Mitsios J V, et al. Eur J Biochem. 2004; 271:855-862). PRP was activated by arachidonic acid (AA; 0.5 mM), Thrombin Receptor Activating Peptide-6 (TRAP-6; 10 μM) and ADP (10 μM), as agonists. Samples were prepared by evaporating the methanol under nitrogen and then by dissolving the residue in DMSO. The maximal aggregation, achieved within 4 min after the addition of each agonist, was determined and expressed as a percentage of 100% light transmission calibrated for each specimen (% maximal aggregation). The inhibitory efficacy of the seeds extract expressed as IC₅₀ value (half maximal inhibitory concentration). All values derived from at least three independent experiments performed for each platelet agonist, using 3 different methanol extract preparations (prepared as described above) and are expressed as the mean±standard deviation (S.D.). The final concentration of DMSO in each assay was 0.01% by volume. As it is shown in FIG. 1, the methanol extract of Acanthus mollis seeds inhibits, in a dose-dependent manner, platelet aggregation induced by AA (IC₅₀=0.15 mg/mL) and TRAP-6 (IC₅₀=0.14 mg/mL), but not by ADP, indicating that its inhibitory effect concerns specific platelet activation pathways, mediated by AA (it is metabolized in platelets by COX-1 to generate TxA₂ which activates platelets via its receptor), and the platelet receptor PAR-1 of thrombin, which is specifically activated by the peptide TRAP-6 used in our experiments.

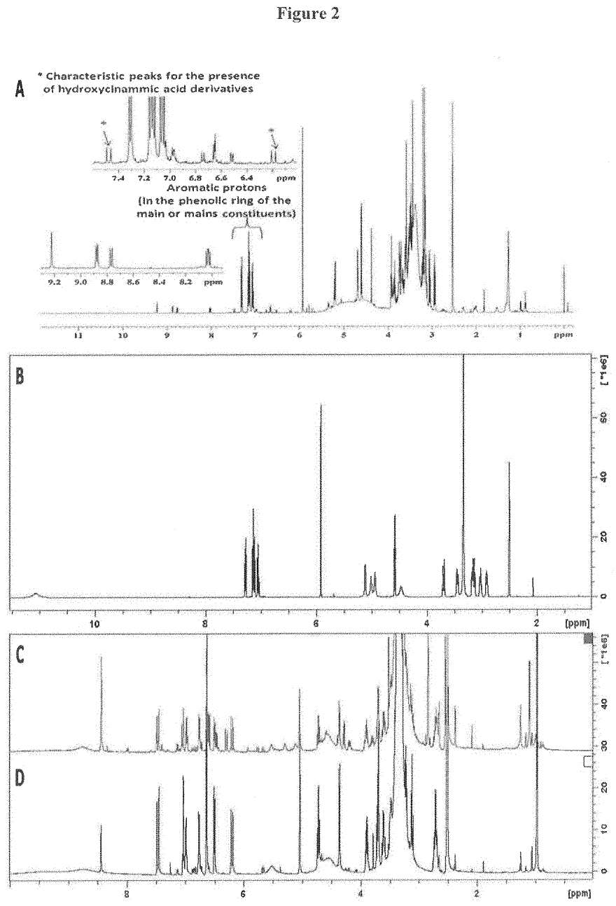

Subsequently, analysis of the phytochemical profile of the methanol extract of Acanthus mollis seeds was performed. The residue derived after methanol evaporation under nitrogen (5 mg) was dissolved in 0.5 mL DMSO-d₆ and transferred to NMR tubes (5 mm), so that 1D ¹H-NMR spectra was recorded and characteristic peaks were indicated (FIG. 2A). Trimethylsilylpropanoic sodium salt (TMSP-d4) used as a reference compound. NMR experiments held at the Center of Nuclear Magnetic Resonance of University of Ioannina, on a Brüker LC-NMR AV-500 spectrometer (Brüker). Experimental conditions were as follows: number of scan (NS)=256, T=298 K, experimental time=30 min, 500 MHz). The NMR system was controlled by the software Top Spin (Brüker). The assessment of results was based on 2D ¹H-¹³C HSQC and HMBC spectra.

LC-MS method was also used for the determination of the major compounds of the methanol extract as a supplementary method of the NMR spectroscopy, and vice versa. Samples from the methanol extract were diluted to reach the concentration of 0.05 mg/mL. Ten μL of filtered sample (0.45 μm) were injected into the LC-MS instrument. All LC-MSⁿ experiments were: performed on a quadrupole ion trap mass analyzer (Agilent Technologies, model MSD trap SL) retrofitted to a 1100 binary HPLC system equipped with an degasser, autosampler, diode array detector and electrospray ionization source (Agilent Technologies, Karlsruhe, Germany). All hardware components were controlled by Agilent Chemstation Software. Separation was achieved on a 15 cm×4.6 mm i.d., 5 μm Zorbax Eclipse XDB-C18 analytical column (Agilent, USA), at a flow rate of 0.6 mL/min, using as solvent A (water/formic acid, 99.9:0.1 v/v) and solvent B (acetonitrile). The gradient used for the analysis was: 0-6 min 90% A, isocratic elution; 6-10 min 95-85% A; 10-15 min 85% A, isocratic elution; 15-20 min 85-75% A; 20-35 min 75-60% A; 35-40 min 60-90% A. The UV/vis spectra were recorded in the range 200-550 nm and chromatograms were acquired at 280 and 330 nm.

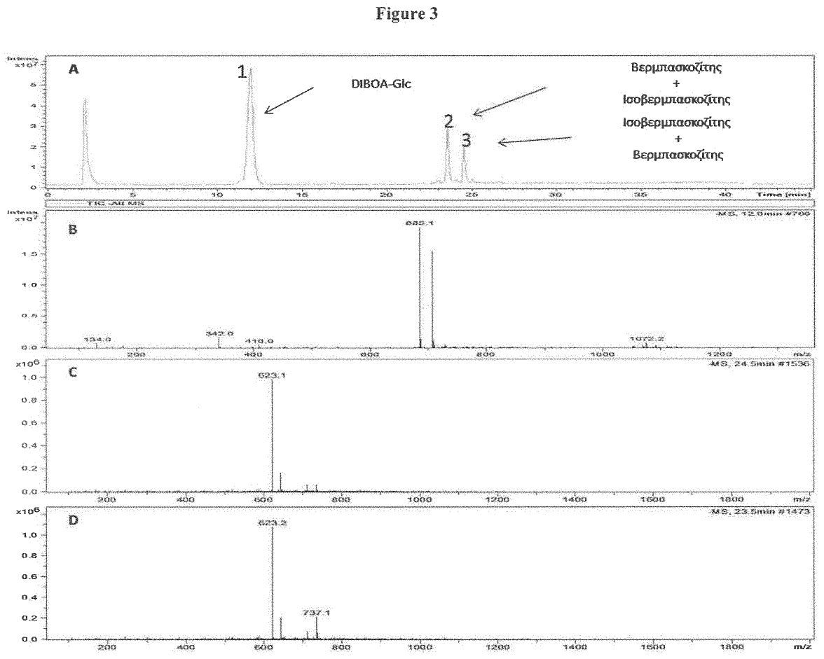

Both precursor and product (MS² and MS³) ions scanning of the phenolic compounds were monitored between m/z 50-m/z 1.200 in negative polarity. The ionization source conditions were as follows: capillary voltage, 3.5 kV; drying gas temperature, 350° C.; nitrogen flow and pressure, 11 L/min and 50 psi, respectively. Maximum accumulation time of ion trap and the number of MS repetitions to obtain the MS average spectra were set at 30 and 3 ms, respectively. The total ion current (TIC) chromatogram (FIG. 3A), UV chromatogram and MS, MS² and MS³ spectra were recorded. Identification of major compounds (1, 2 and 3) was based on accurate mass measurements of the pseudomolecular [M−H]⁻ ions and their fragmentation pattern, as it has been previously described (Wolf R B, et al. J Nat Prod. 1985; 48:59-63; Ryan D, et al. J Chromatogr. A 1999; 832:87-96). According to the results, peak 1 contains the compound 2-O-β-D-glucopyranosyl-4-hydroxy-1,4-benzoxazin-3-one (dimer) (DIBOA-Glc), peak 2 contains mainly Verbascoside and traces of Isoverbascoside (Isoacteoside), whereas peak 3 contains mainly Isoverbascoside and traces of Verbascoside. All characteristics of these compounds are summarized in the following table.

TABLE

| | | | −MS² | −MS³ | |
|---|---|---|---|---|---|
| | Rₜ | [M − H]⁻ | [M − H]⁻ | [base peak] | |
| Peak | (min) | (m/z) | (m/z) (%) | (m/z) (%) | Compounds |
| 1 | 12.0 | 685 (100), | 342 (100) | 134 (100), | DIBOA-Glc |
| | | 342 (12) | | 180 (28), | |
| | | | | 162 (15) | |
| 2 | 23.6 | 623 (100) | 461 (100) | 135 (100), | Verbascoside, |
| | | | | 315 (99), | Isoverbascoside |
| | | | | 161 (16), | (in traces) |
| | | | | 297 (19) | |
| 3 | 24.6 | 623 (100) | 461 (100), | 135 (100), | Isoverbascoside, |
| | | | | 315 (96), | Verbascoside |
| | | | 315 (3) | 161 (12), | (in traces) |
| | | | | 297 (12) | |

In order to ascertain the identity of the major compounds, these were isolated by using preparative High Pressure Liquid Chromatography (HPLC). The residue derived after methanol evaporation under nitrogen (20-60 mg) was again dissolved in methanol (3.2 mL). 3.1 mL of filtered sample (0.45 μm) was injected into the HPLC instrument (preparative Chromatography system, Shimadzu, Japan) and separation was achieved on 25 cm×21.2 mm i.d., 10 μm Ascentis C18 analytical column (Shimadzu, Japan), at a flow rate of 16 mL/min, using as solvent A (water/formic acid, 99.9:0.1 v/v) and solvent B (acetonitrile). The gradient used for the analysis was the same as described in the LC-MS experiments and the UV/vis spectra were acquired at 280 and 330 nm. The fractions were lyophilized before they used in subsequent experiments.

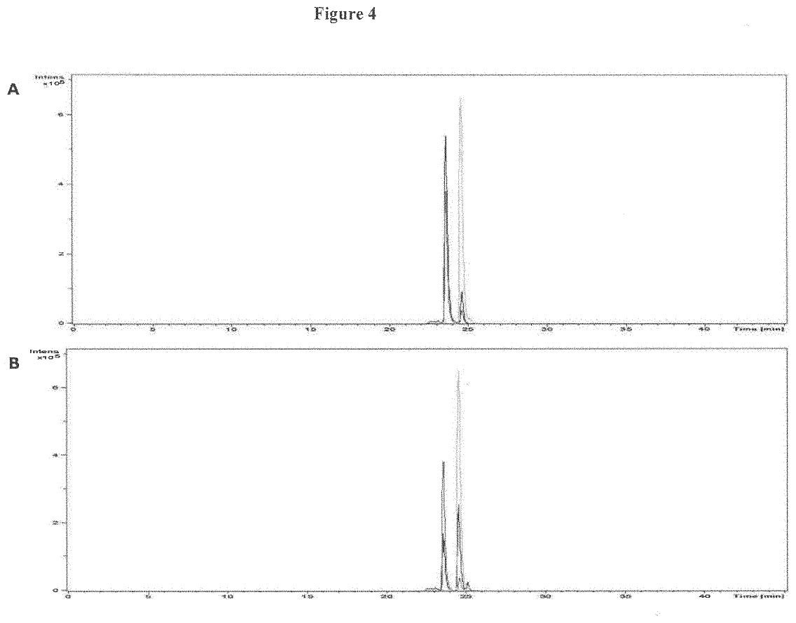

Three fractions were isolated using the above HPLC methodology and were then analyzed by LC-MS using the same experimental conditions described above. TIC chromatograms, UV chromatograms and MS, MS² and MS³ spectra were recorded. According to our results, Fraction 1 corresponds to DIBOA-Glc (FIG. 3B), while Fraction 2 (FIG. 3C) and Fraction 3 (FIG. 3D) are a mixture of Verbascoside and Isoverbascoside, the Verbascoside been in greater quantity over Isoverbascoside in Fraction 2 and Isoverbascoside been in greater quantity over Verbascoside in Fraction 3. In order to ascertain the presence of Verbascoside and Isoverbascoside, authentic standard compounds (Medchem Express, USA) were used to the desired concentration of 0.05 mg/mL (FIG. 4).

Additionally, the isolated by HPLC fractions were submitted to analysis by NMR. Each fraction (5 mg) was dissolved in 0.5 mL DMSO-d6 and transferred to NMR tubes (5 mm), so that 1D ¹H-NMR spectra were recorded. Trimethylsilylpropanoic sodium salt (TMSP-d4) was used again as reference compound. The complete assignment of Fraction 1 as DIBOA-Glc (FIG. 2B), Fraction 2 (FIG. 2C) and Fraction 3 (FIG. 2D) as a mixture of Verbascoside and Isoverbascoside was in accordance with previously published values (Hartenstein H, et al. Phytochemistry. 1994; 35:827-828.; Kanchanapoom T, et al. Phytochemistry. 2011; 58:637-640.; Yin H, et al. J Chromatogr. A. 2008; 1205:177-181).

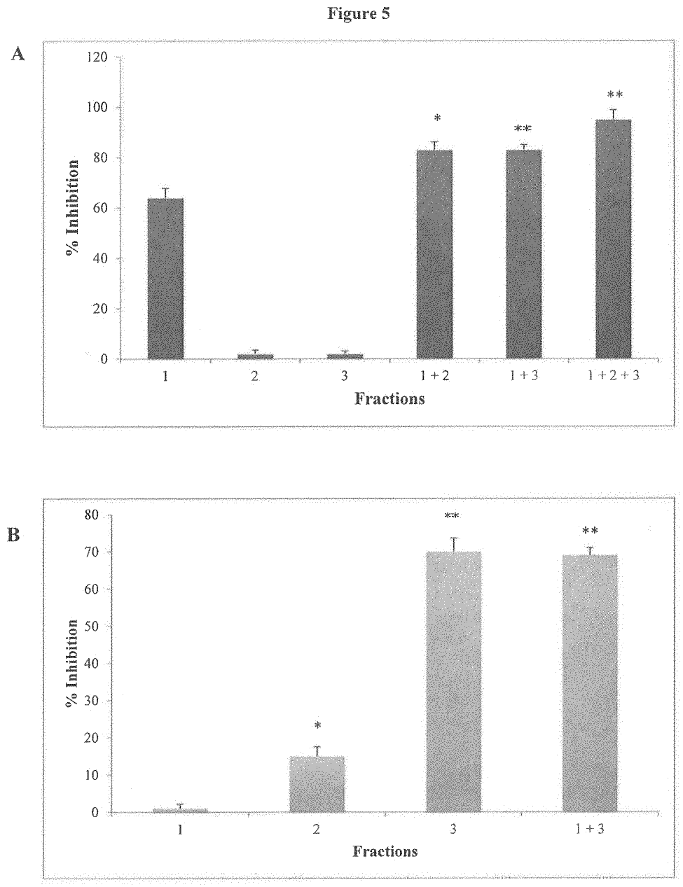

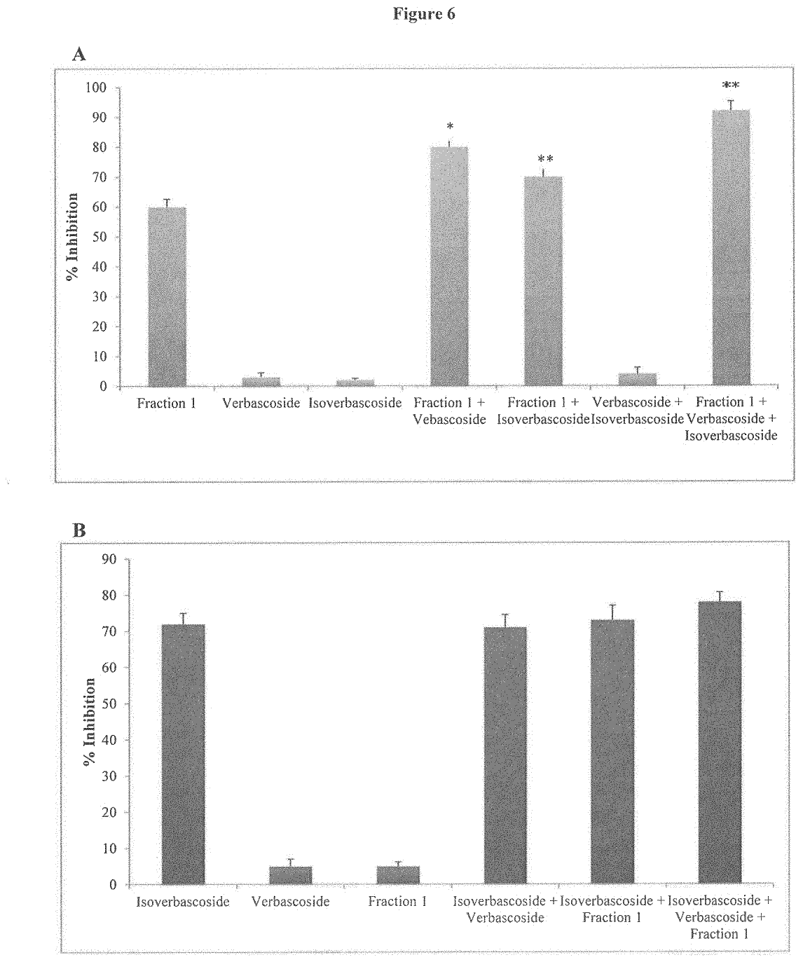

Following the above analysis, the biological effect of the three fractions towards platelet aggregation in vitro was studied in PRP by the LTA as we described above, using AA (0.5 mM) and TRAP-6 (10 μM) as agonists, since the total extract inhibited platelet aggregation induced only by these 2 agonists (FIG. 1). Each fraction was dissolved in DMSO and used at a final concentration of 1 mg/mL. Standards of Verbascoside and Isoverbascoside were also dissolved in DMSO and used at a final concentration of 1 mg/mL. As it is shown in FIG. 5A, among the 3 fractions, only fraction 1 (DIBOA-Glc) inhibited platelet aggregation induced by AA, exhibiting a threshold concentration (the lower concentration that induces the maximum inhibitory effect) of 0.2 mg/mL. This concentration was not much lower to that of the total extract 0.25 mg/mL (as it would expected, since we used a purified fraction from the total extract that expresses the inhibitory activity towards AA-induced platelet aggregation). Therefore, we used a combination of fraction 1 with fraction 2 or 3 (at mass ratios of 1:1) or a combination of the 3 fractions (at mass ratios of 1:1:1). As it is shown in FIG. 5A, fractions 2 or 3 do not inhibit platelet aggregation, however, they significantly increased the antiplatelet effect of fraction 1, the maximum inhibition been observed when the combination of 3 fractions was used. This suggests that Verbascoside and Isoverbascoside express a synergistic inhibitory effect with DIBOA-Glc towards AA-induced platelet aggregation. To further support this suggestion, we studied the effect of standards Verbascoside and Isoverbascoside in combination with fraction 1 (DIBOA-Glc). As it is shown in FIG. 6A, Verbascoside and Isoverbascoside alone or their combination do not inhibit platelet aggregation towards AA-induced platelet aggregation but significantly increased the antiplatelet effect of fraction 1 (DIBOA-Glc), the maximum inhibition been observed when their combination was added to fraction 1.

Next we evaluated the biological effect of the three fractions towards platelet aggregation induced by TRAP-6. As it is shown in FIG. 5B, inhibitory activity was expressed mainly by Fraction 3 (containing primarily Isoverbascoside and traces of Verbascoside), whereas a much lower activity was express by fraction 2 (containing primarily Verbascoside and traces of Isoverbascoside). Fraction 1 had no inhibitory activity towards platelet aggregation induced by TRAP-6, neither it increased the inhibitory activity expressed by fraction 3 (FIG. 5B). These results suggest that the inhibitory effect of the total methanol extract towards TRAP-6-induced platelet aggregation is primarily attributed to Isoverbascoside. To further support this suggestion, we studied the effect of standards Isoverbascoside and Verbascoside alone or their combination (at mass ratio 1:1) as well as in combination with fraction 1 (DIBOA-Glc) (at mass ratio 1:1:1). As it is shown in FIG. 6B, only Isoverbascoside inhibited platelet aggregation, whereas Verbascoside or fraction 1 (DIBOA-Glc) or their combination, failed to increase Isoverbascoside's inhibitory effect.

Overall, the methanol extract of Acanthus mollis seeds, inhibits platelet aggregation mediated through AA pathway and PAR-1 receptor. The inhibitory effect towards AA is primarily attributed to its DIBOA-Glc content, whereas a synergistic effect is observed by the combination of DIBOA-Glc with Isoverbascoside, Verbascoside as well as their combination. By contrast, the inhibitory effect towards platelet aggregation mediated through PAR-1 receptor is exclusively attributed to its Isoverbascoside content.

To date, patients with cardiovascular disease should receive antiplatelet therapy, primarily aspirin (inhibits COX-1 and therefore AA-induced platelet aggregation) and an ADP receptor P2Y12 antagonist (clopidogrel, prasugrel or ticagrelor) (Kalantzi K I, et al. Expert Rev Clin Pharmacol. 2012; 5:319-36; Tsoumani M E, Tselepis A D, Curr Pharm Des. 2017; Epub ahead of print)). Several studies however have demonstrated that some patients receiving aspirin or clopidogrel do not adequately respond to the drug action and exhibit a new ischemic cardiovascular event (a phenomenon named as aspirin or clopidogrel resistance). Recent studies showed that a PAR-1 antagonist (vorapaxar) exhibit an important clinical efficacy in preventing an atherothrombotic event, however its therapeutic usefulness has been limited due to the increase in bleeding risk (Moschonas I C, et al. Int J Cardiol. 2015; 185:9-18).

Therefore, based on the present results, the constituents of Acanthus mollis seeds described above may be considered as new therapeutic agents to be used in CVD patients, since they can replace aspirin in aspirin resistant patients (DIBOA-Glc alone or in combination with Verbascoside or/and Isoverbascoside) and also to be used as a specific antagonist of the platelet thrombin receptor PAR-1 (Isoverbascoside). Finally, the total extract could be used as an important supplement in addition to the standard therapy with aspirin and a P2Y12 antagonist in these patients.

### BRIEF DESCREPTION OF SHEMES

FIG. 1 shows representative dose-response curves for the inhibitory effect of the methanol extract of Acanthus mollis seeds on platelet aggregation induced by A.A (0.5 mM) (A) and TRAP-6 (10 μM) (B) but not by ADP (10 μM) (C). The final concentration of DMSO was 0.01% by volume.

FIG. 2 shows the 1D ¹H-NMR spectrum of the methanol extract of Acanthus mollis seeds (A), fraction 1 (B), fraction 2 (C) and fraction 3 (D) in DMSO-d₆ (NS=256, T=298K, experimental time=30 mM, 500 MHz). As it can be observed in FIGS. 2C and 2D fractions 2 and 3 contain a mixture of Verbascoside and Isoverbascoside.

FIG. 3 shows (A) the TIC chromatogram of A. mollis methanolic extract where the main components are indicated, (B) the MS spectrum of fraction 1 (m/z 685.1, Rₜ=11.9 min), (C) the MS spectrum of fraction 2 (m/z 623.2, Rₜ=23.6 min) and (D) the MS spectrum of fraction 3 (m/z 623.2, Rₜ=24.5 min).

FIG. 4 shows an overlaid total ion chromatograms of (A) fraction 2 (black), standard Verbascoside (red) and Isoverbascoside (green), demonstrating the existence of verbascoside and a small quantity of isoverbascoside in fraction 2 and (B) fraction 3 (black), standard Verbascoside (red) and Isoverbascoside (green), demonstrating the existence of Isoverbascoside and a small quantity of Verbascoside in fraction 3.

FIG. 5 is a bar-graph demonstrating the effect of fractions 1,2 and 3 as well as their combinations on human platelet aggregation in PRP induced by (A) AA (0.5 mM) and (B) TRAP-6 (10 μM). The final concentration of each fraction in PRP was 1 mg/mL. The final concentration of DMSO in PRP was 0.01% by volume. *P<0.05 and **P<0.01 compared with fraction 1.

FIG. 6 is a bar-graph demonstrating the effect of fraction 1, Verbascoside and Isoverbascoside as well as their combinations on human platelet aggregation in PRP induced by (A) AA (0.5 mM) and (B) TRAP-6 (10 μM). The final concentration in PRP of each agent used was 1 mg/mL. The final concentration of DMSO was 0.01% by volume. *P<0.05 and **P<0.01 compared with fraction 1.

1-6. (canceled)

7. Use of a total methanolic extract of Acanthus mollis seeds for preparation of a medicament or a supplement for human use having a dual antiplatelet and, therefore, antithrombotic activity by specifically inhibiting an AA-pathway and a PAR-1 receptor pathway.

8. Use of DIBOA-Glc for preparing a medicament for human use having an antiplatelet effect and, therefore, antithrombotic activity by specifically inhibiting an arachidonic acid-induced platelet aggregation.

9. Use of Isoverbascoside for preparing a medicament for human use having a specific inhibitory action towards platelet aggregation mediated through a PAR-1 receptor.

10. Use of a combination of DIBOA-Glc with Isoverbascoside or Verbascoside, or both, for preparing a medicament for human use exhibiting a synergistic effect towards arachidonic acid-induced platelet aggregation.
